SEQUENCE LISTING (12 sequences)

SEQ ID NO: 1 - DNA, 394 bp, Artificial Sequence
  Col2a1 promoter
cccgggtttggggggcaggggccatctctgcgccccgcccgatcaggccactcggcgcactaggggtggagggcgggaagcgtgactcccagagaggggggtccggcttgggcaggtgcgggcactggcagggcccaggcgggctccgggggcgggcggttcaggttacagcccagctgggggcagggggcggcccgcggtttgggcgagttcgccagcctcgaaaggggccgggcgcatataacgggcgccgcggcggggagaagacgcagagcgctgctgggctgccgggtctcccgcttcctcctcctgctccaagggcctcctgcatgagggcgcggtagagacccggacccgcgccgtgctcctgccgtttcgctgcgctccgcccggg

SEQ ID NO: 2 - DNA, 725 bp, Artificial Sequence
  Col2a1 Promoter Enhancer
gaattcggccacacccctcctctccatctttccacgagtttgagaaacttactggcggcggcgtctttgaccctcatctgcatttcagagccctcgcctccgaaagtgcccctggctcaggggagagatctcaatcctcctttgtgaggcttgtttgcattgggagattggcagcgatggcttccagatggggctgaaacgctgcccgtatttatttaaactggttcctcgcggagacctgtgaatcgggctctgtgtgcgctcgagaaaagccccattcatgagagacgaggtccagtgggttctctcgtactcccagaccccctctcccacaatgcccccctgtgcccgcccgccgccacctctcggctccagccctgcgcagagcggcggtgaagcaaaacagttccccgaaagaggtagctttttaattggcttgccacaaagaatcacttatacggccctgcggtaatgaggggaaccggatcaggcgcgccgggatgctatcggcagccgttttggagcagcaattatggtggtgctgggctcctccgtccacacctaggggatccggttacggcgctggctcctttctggggcagtcatttaatcccacttttcactctcccggtgtctgtgagcgagccgtgtccagagccgcagccacagagtcactcagcggctcttacacccagcgcagcctggccccgcccctgcagcccggg

SEQ ID NO: 3 - DNA, 8884 bp, Artificial Sequence
  pCDH-ENS-C2P-copGFP-Puro
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggccacacccctcctctccatctttccacgagtttgagaaacttactggcggcggcgtctttgaccctcatctgcatttcagagccctcgcctccgaaagtgcccctggctcaggggagagatctcaatcctcctttgtgaggcttgtttgcattgggagattggcagcgatggcttccagatggggctgaaacgctgcccgtatttatttaaactggttcctcgcggagacctgtgaatcgggctctgtgtgcgctcgagaaaagccccattcatgagagacgaggtccagtgggttctctcgtactcccagaccccctctcccacaatgcccccctgtgcccgcccgccgccacctctcggctccagccctgcgcagagcggcggtgaagcaaaacagttccccgaaagaggtagctttttaattggcttgccacaaagaatcacttatacggccctgcggtaatgaggggaaccggatcaggcgcgccgggatgctatcggcagccgttttggagcagcaattatggtggtgctgggctcctccgtccacacctaggggatccggttacggcgctggctcctttctggggcagtcatttaatcccacttttcactctcccggtgtctgtgagcgagccgtgtccagagccgcagccacagagtcactcagcggctcttacacccagcgcagcctggccccgcccctgcagcccgggtttggggggcaggggccatctctgcgccccgcccgatcaggccactcggcgcactaggggtggagggcgggaagcgtgactcccagagaggggggtccggcttgggcaggtgcgggcactggcagggcccaggcgggctccgggggcgggcggttcaggttacagcccagctgggggcagggggcggcccgcggtttgggcgagttcgccagcctcgaaaggggccgggcgcatataacgggcgccgcggcggggagaagacgcagagcgctgctgggctgccgggtctcccgcttcctcctcctgctccaagggcctcctgcatgagggcgcggtagagacccggacccgcgccgtgctcctgccgtttcgctgcgctccgcccgggtctagagctagcatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccaaatcggagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 4 - DNA, 9787 bp, Artificial Sequence
  pCDH-ENS-C2P-fLuc-Puro
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggccacacccctcctctccatctttccacgagtttgagaaacttactggcggcggcgtctttgaccctcatctgcatttcagagccctcgcctccgaaagtgcccctggctcaggggagagatctcaatcctcctttgtgaggcttgtttgcattgggagattggcagcgatggcttccagatggggctgaaacgctgcccgtatttatttaaactggttcctcgcggagacctgtgaatcgggctctgtgtgcgctcgagaaaagccccattcatgagagacgaggtccagtgggttctctcgtactcccagaccccctctcccacaatgcccccctgtgcccgcccgccgccacctctcggctccagccctgcgcagagcggcggtgaagcaaaacagttccccgaaagaggtagctttttaattggcttgccacaaagaatcacttatacggccctgcggtaatgaggggaaccggatcaggcgcgccgggatgctatcggcagccgttttggagcagcaattatggtggtgctgggctcctccgtccacacctaggggatccggttacggcgctggctcctttctggggcagtcatttaatcccacttttcactctcccggtgtctgtgagcgagccgtgtccagagccgcagccacagagtcactcagcggctcttacacccagcgcagcctggccccgcccctgcagcccgggtttggggggcaggggccatctctgcgccccgcccgatcaggccactcggcgcactaggggtggagggcgggaagcgtgactcccagagaggggggtccggcttgggcaggtgcgggcactggcagggcccaggcgggctccgggggcgggcggttcaggttacagcccagctgggggcagggggcggcccgcggtttgggcgagttcgccagcctcgaaaggggccgggcgcatataacgggcgccgcggcggggagaagacgcagagcgctgctgggctgccgggtctcccgcttcctcctcctgctccaagggcctcctgcatgagggcgcggtagagacccggacccgcgccgtgctcctgccgtttcgctgcgctccgcccgggtctagagctagcatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcgttccggatactgcgattttaagtgttgtcccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgctcgagtgatgagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccaaatcggagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 5 - DNA, 20 bp, Artificial Sequence
  Forward primer of Col2a1
aaccagattgagagcatccg

SEQ ID NO: 6 - DNA, 18 bp, Artificial Sequence
  Reverse primer of Col2a1
accttcatggcgtccaag

SEQ ID NO: 7 - DNA, 17 bp, Artificial Sequence
  Forward primer of Sox9
acttgcacaacgccgag

SEQ ID NO: 8 - DNA, 21 bp, Artificial Sequence
  Reverse primer of Sox9
ctggtacttgtaatccgggtg

SEQ ID NO: 9 - DNA, 20 bp, Artificial Sequence
  Forward primer of Col10a1
acgataccaaatgcccacag

SEQ ID NO: 10 - DNA, 22 bp, Artificial Sequence
  Reverse primer of Col10a1
gtaccttgctctcctcttactg

SEQ ID NO: 11 - DNA, 19 bp, Artificial Sequence
  Forward primer of Gapdh
acatcgctcagacaccatg

SEQ ID NO: 12 - DNA, 22 bp, Artificial Sequence
  Reverse primer of Gapdh
tgtagttgaggtcaatgaaggg

(19) United States
(12) Patent Application Publication
(10) Pub. No.: US 2023/0107128 A1
(43) Pub. Date: Apr. 6, 2023

(54) SCREENING SYSTEM FOR HUMAN-TRANSFORMED CARTILAGE CELL LINE-BASED CARTILAGE DISEASE TREATMENT AGENTS

(71) Applicant: LUGEN SCI CO., LTD., Bucheon-si, Gyeonggi-do (KR)

(72) Inventor: Jong Min LEE, Goyang-si (KR)

(73) Assignee: LUGEN SCI CO., LTD., Bucheon-si, Gyeonggi-do (KR)

(21) Appl. No.: 17/273,949

(22) PCT Filed: Sep. 3, 2019
(86) PCT No.: PCT/KR2019/011355

§ 371 Date: Mar. 5, 2021

(30) Foreign Application Priority Data
Sep. 5, 2018 (KR) 10-2018-0106190
Aug. 28, 2019 (KR) 10-2019-0105987

(51) Int. Cl. C12N 15/10; G01N 33/50

(52) U.S. Cl. CPC C12N 15/1055; G01N 33/5044

(57) ABSTRACT

A recombination expression vector can be used for screening cartilage disease treatment agents. A cell line is transformed using the expression vector. A method using the foregoing screen drugs that are effective in treating cartilage diseases, and since all constituent factors are composed of human-derived genetic factors, new drugs selected through this system are considered to be more effective in treating human cartilage diseases. Furthermore, using additional transformation, it can be evaluated whether genes having unknown functions can be used to treat cartilage diseases. This establishes the ability to not only compare the cartilage disease treatment functions of various drugs, but also to evaluate the optimal treatment concentration and indirect cytotoxicity. Moreover, 2-anthraquinonecarboxylic acid, which is a novel substance having cartilage regeneration efficacy discovered through the screening system, is utilizable in the treatment of various cartilage disease.

### TECHNICAL FIELD

The present invention relates to a recombinant expression vector for screening cartilage disease treatment agents, a cell line transformed using the expression vector, and a method for using the same to screen drugs that are effective in treating cartilage diseases.

This application claims the benefits of Korean Patent Application No. 10-2018-0106190 filed on Sep. 5, 2018 and Korean Patent Application No. 10-2019-0105987 filed on Aug. 28, 2019 with the Korean Intellectual Property Office, of which all contents disclosed in the specification and drawings of the application are incorporated herein by reference in its entirety.

### BACKGROUND ART

Damage to articular cartilage is not regenerated into its original tissues and thus is difficult to heal. Accordingly, many attempts have been made to solve this problem. With regard to advanced degenerative arthritis, its standard treatment has been to remove the affected cartilage and bone and perform an artificial joint replacement made of metal and polyethylene. However, the life of the artificial joint has become a problem, when the procedure is performed on relatively young patients in their 60s or under. Such cartilage damage is caused by osteoarthritis that causes a traumatic defect or gradual destruction to the joint cartilage tissues and its frequency is very high. However, the damaged cartilage is difficult to regenerate into hyaline cartilage bone, which is an original tissue of the cartilage, and the molecular control mechanism of cartilage regeneration has not been known yet.

Conventional therapies for injured joints include classical drug treatment, autologous osteochondral transplantation, bone marrow perforation, artificial joint replacement, and the like, out of which the classical drug treatment corresponds to a conservative treatment. However, this treatment alleviates symptoms only and thus is limited to restricted recovery of functions. The autologous osteochondral transplantation, which is used for traumatic defects of articular cartilage, causes damage to the donor site due to the collection of bone-cartilage fragments, and has the disadvantage of limiting the amount of collection. In addition, the bone marrow perforation performed for moderately advanced osteoarthritis regenerates fibrocartilage instead of hyaline cartilage bone, which is the original cartilage tissue, and has a disadvantage of poor clinical results. The artificial joint replacement, which is a standard treatment method for osteoarthritis, also has a problem with the life of the artificial joint, when it is performed on relatively young patients.

Meanwhile, stem cells for regenerating articular cartilage are characterized by their self-renewal ability and differentiation potency into cells constituting specific tissues, and are recently proposed as a new cell source for application to articular cartilage treatment. Thus, theoretically, those stem cells have a chance of overcoming the limits of the existing cell therapy using chondrocytes and being applied even to the overall degeneration and damage of the articular cartilage. In addition, adult mesenchymal stem cells and mesenchymal progenitor cells have the advantage of causing neither ethical problem nor in vivo rejection reaction during homograft.

However, not all adult mesenchymal stem cells are completely differentiated into chondrocytes at the same time. Thus, there is a need for a method of inducing their differentiation into homogeneous chondrocytes. And, in order to apply the chondrocytes differentiated from the stem cells to cell therapy, there is need for a method of precisely controlling and inhibiting hypertrophy, which is a premonitory symptom of apoptosis and osteogenic differentiation of cells induced into the cartilage when mesenchymal stem cells are differentiated into the cartilage.

In recent years, compared to stem cell transplantation, which has been rapidly growing as a therapy for articular cartilage, classical drug treatment has remained only as conservative treatment until now probably because of the absence of a system capable of screening only novel drugs effective for damaged joints compared to a highly developed and rapid drug separation process. Considering the fact that more than 90% of drugs in clinical trials fail to enter the market due to side effects not found in animal studies or lack of validity of drug effects, more and more importance has been put on developing a test system capable of more effectively screening synthetic or natural compounds in terms of efficacy and problems.

Meanwhile, as aging continues, humans naturally develop degenerative arthritis, which is caused by a decrease in the amount of type II collagen synthesis due to aging of chondrocytes. In normal joints, type II collagen is a component of the extracellular matrix (ECM), which forms the basis of 85-90% of the articular cartilage. Thus, arthritis occurs when the synthesis of type II collagen becomes insufficient or inhibited from chondrocytes present in the articular cartilage. On the contrary, the genetic and non-genetic drugs effective in maintaining and regenerating healthy articular cartilage are to have an effect on performing the function of finally normalizing the synthesis of type II collagen or inducing overexpression thereof. For example, until recently, the SOX-trio gene, transforming growth factor beta (TGF-beta), dexamethasone, Wnt inhibitors, etc., have been known as essential factors for maintaining the shape of chondrocytes and inducing the differentiation of stem cells into chondrocytes. With regard to a beneficial role of forming the cartilage, these genetic factors and chemical organic compounds ultimately aim at synthesizing a large amount of type II collagen.

Thus, the development of a system capable of evaluating drugs capable of enhancing the synthesis of type II collagen is expected to facilitate the development of excellent arthritis treatment agents, and thus there is a growing demand for many studies in this regard.

### DISCLOSURE

### Technical Problem

Based on the fact that most of the collagen present in healthy articular cartilage is composed of type II collagen, the present inventors have developed a screening system for human-transformed cartilage cell line-based cartilage disease treatment agents so as to screen drugs capable of directly or indirectly controlling an expression of the type II collagen and have experimentally confirmed an actual effect, thereby completing the present invention.

Accordingly, an object of the present invention is to provide a recombinant expression vector for screening cartilage disease treatment agents, including Col2a1 promoter (C2P), Col2a1 promoter enhancer (ENS), and a reporter gene.

Another object of the present invention is to provide a transformed cell line for screening cartilage disease treatment agents transformed using the recombinant expression vector.

Still another object of the present invention is to provide a method for screening cartilage disease treatment agents, the method comprising: treating the transformed cell line with a candidate drug; and measuring a level of expression or activity of a reporter gene in the transformed cell line treated with the candidate drug.

In addition, an object of the present invention is to provide a composition for treating cartilage diseases, containing 2-anthraquinonecarboxylic acid or derivatives thereof as an effective ingredient.

Furthermore, an object of the present invention is to provide a method for treating cartilage diseases, the method comprising administering a composition containing 2-anthraquinonecarboxylic acid or derivatives thereof as an effective ingredient to an individual.

Moreover, an object of the present invention is to provide a use of a composition containing 2-anthraquinonecarboxylic acid or derivatives thereof as an effective ingredient for treating cartilage diseases.

Besides, an object of the present invention is to provide a use of 2-anthraquinonecarboxylic acid or derivatives thereof for producing a drug used for treating cartilage diseases.

However, the technical objects to be achieved by the present invention is not limited to those mentioned above, and other objects that are not mentioned will be clearly understood by those skilled in the art from the following description.

### Technical Solution

To achieve the object of the present invention as above, the present invention may provide a recombinant expression vector for screening cartilage disease treatment agents, including Col2a1 promoter (C2P), Col2a1 promoter enhancer (ENS), and a reporter gene.

In one embodiment of the present invention, the Col2a1 promoter may be represented by a sequence of SEQ ID NO: 1.

In another embodiment of the present invention, the Col2a1 promoter enhancer may be represented by a sequence of SEQ ID NO: 2.

In still another embodiment of the present invention, the Col2a1 promoter may be human type II collagen promoter.

In still another embodiment of the present invention, the Col2a1 promoter and the Col2a1 promoter enhancer may be derived from human mesenchymal stem cells.

The mesenchymal stem cells may be derived from bone marrow without limitation.

In still another embodiment of the present invention, the reporter gene may be a gene encoding luciferase or green fluorescent protein (GFP).

In still another embodiment of the present invention, the recombinant expression vector may be prepared by using a lentiviral vector.

In still another embodiment of the present invention, the recombinant expression vector may be represented by a sequence of SEQ ID NO: 3.

In still another embodiment of the present invention, the recombinant expression vector may be represented by a sequence of SEQ ID NO: 4.

In addition, the present invention may provide a transformed cell line for screening cartilage disease treatment agents transformed with the lentiviral vector prepared by using the recombinant expression vector.

In one embodiment of the present invention, the transformed cell line may be a human cartilage cell line.

In another embodiment of the present invention, the transformed cell line may be C28/I2-EC2P-fLuc-CN5 of accession number KCLRF-BP-00456.

In addition, the present invention may provide a method for screening cartilage disease treatment agents, the method comprising: treating the transformed cell line with a candidate drug; and measuring a level of expression or activity of a reporter gene in the transformed cell line treated with the candidate drug.

In one embodiment of the present invention, the reporter gene may be a gene encoding luciferase or green fluorescent protein (GFP).

In another embodiment of the present invention, the method may further include determining that a drug having a higher level of expression or activity of the reporter gene measured above is regarded to have a more excellent regenerative activity for damaged cartilage.

In addition, the present invention may provide a composition for treating cartilage diseases, containing 2-anthraquinonecarboxylic acid or derivatives thereof as an effective ingredient.

In one embodiment of the present invention, the composition may treat cartilage diseases through cartilage regeneration.

In another embodiment of the present invention, the cartilage disease may be selected from the group consisting of degenerative arthritis, rheumatoid arthritis, fracture, damage to muscle tissues, plantar fasciitis, humerus epicondylitis, calcification myositis, nonunion of fracture, and joint damage caused by injury.

In addition, the present invention may provide a method for treating cartilage diseases, the method including administering a composition containing 2-anthraquinonecarboxylic acid or derivatives thereof as an effective ingredient to an individual.

Furthermore, the present invention may provide a use of a composition containing 2-anthraquinonecarboxylic acid or derivatives thereof as an effective ingredient for treating cartilage diseases.

Moreover, the present invention may provide a use of 2-anthraquinonecarboxylic acid or derivatives thereof for producing a drug used for treating cartilage diseases.

### Advantageous Effects

The screening system for cartilage disease treatment agents according to the present invention uses human-derived factors as all of the components, and thus the novel drugs selected through this system can be considered to be more effective in treating cartilage diseases in humans. In addition, the present invention has the advantage of evaluating the therapeutic effects of genes having unknown functions on cartilage diseases through additional transformation, and thus is capable of not only comparing the therapeutic effects of various drugs on cartilage diseases with each other, but also evaluating the optimal treatment concentration and the presence of indirect cytotoxicity. In addition, the present invention is capable of screening at the same time. Furthermore, 2-anthraquinonecarboxylic acid, which has been found through the screening system of the present invention, shows an excellent efficacy of regenerating cartilage, and thus may be used as a therapeutic agent for various cartilage diseases.

### DESCRIPTION OF DRAWINGS

FIG. 1 shows a method for preparing a recombinant expression vector for screening cartilage disease treatment agents.

FIG. 2 shows a structure of recombinant expression vector pCDH-ENS-C2P-copGFP-Puro.

FIG. 3 shows a structure of recombinant expression vector pCDH-ENS-C2P-fLuc-Puro.

FIG. 4 shows the results of observing GFP fluorescence to confirm if human type II collagen fusion promoter is expressed specifically to cartilage tissues.

FIG. 5 shows the results of experimenting on the activity of luciferase to select a mono clone of human-transformed cartilage cell line capable of expressing luciferase specifically to cartilage tissues.

FIG. 6 shows the results of experimenting on C28/I2-EC2P-fLuc-CN5 cell line treated with Kartogenin (A of FIG. 6) and TD-198946 (B of FIG. 6), which are drugs for cartilage treatment, at each concentration.

FIG. 7 shows an image of results of C28/I2-EC2P-fLuc-CN5 cell line treated repeatedly four times with Kartogenin and TD-198946, which are drugs for cartilage treatment.

FIG. 8 shows the results of measuring the cartilage regeneration activity of 37 kinds of natural products by using the screening system according to the present invention.

FIG. 9 shows the results of measuring the cartilage regeneration activity of Kartogenin (left graph) and 2-anthraquinonecarboxylic acid (right graph) at each concentration of treatment.

FIGS. 10A and 10B show the results of measuring each cytotoxicity of Kartogenin and 2-anthraquinonecarboxylic acid in C28/I2 cell line (FIG. 10A) and chondrocytes isolated from osteoarthritis patients (FIG. 10B).

FIG. 11 shows the results of comparing the expression patterns of cartilage differentiation markers according to treatment with Kartogenin or 2-anthraquinonecarboxylic acid.

### MODE FOR INVENTION

Based on the fact that most of the collagen present in healthy articular cartilage is composed of type II collagen, the present inventors have developed a screening system for transformed cell lines so as to screen drugs capable of directly or indirectly controlling an expression of the type II collagen and have experimentally confirmed an actual effect, thereby completing the present invention.

In one embodiment of the present invention, it was confirmed that a lentiviral vector containing a type II collagen promoter and a reporter gene may be prepared to show a cartilage-specific expression (see Examples 1 and 2).

In another embodiment of the present invention, as a result of screening a mono clone of human-transformed cartilage cell line capable of expressing luciferase specifically to chondrocytes, it was confirmed that Clone Number (CN) 5 cell line shows a distinct difference in a degree of reactivity to each drug while showing higher luciferase activity compared to other clones (see Example 3).

In another embodiment of the present invention, as a result of verifying the effect of cartilage treatment drugs by using the established C28/I2-EC2P-fLuc-CN5 cell line, it was confirmed that it is possible to directly compare the drug effects, indirectly identify the presence of cytotoxicity, and screen a number of drugs without a repeated test (see Example 4).

In another embodiment of the present invention, the screening system for cartilage disease treatment agents according to the present invention was used to screen 2-anthraquinonecarboxylic acid, which is a novel compound having a cartilage regeneration efficacy, out of 37 types of anti-inflammatory natural compounds, and confirm that the compound has a high cartilage regeneration activity and an elevated cell growth rate (see Example 5).

Accordingly, the present invention may provide a recombinant expression vector for screening cartilage disease treatment agents, including Col2a1 promoter (C2P), Col2a1 promoter enhancer (ENS), and a reporter gene.

As used herein, the term “vector” refers to a DNA product containing a DNA sequence operatively linked to a suitable regulatory sequence capable of expressing DNA in a suitable host. The vector may be a plasmid, a phage particle, or simply a potential genomic insert. Once transformed into an appropriate host, the vector may replicate itself and function independently of the host genome, or in some cases may be integrated into the genome itself. Since the plasmid is now the most commonly used form of vectors, “plasmid” and “vector” are sometimes used interchangeably in the specification of the present invention. However, the present invention may include other forms of vectors that have functions equivalent to those known or to be known in the art, and the “transformation” or “transduction” means that DNA is introduced into a host, so that the introduced DNA may be expressed as an extrachromosomal factor or by completion of chromosomal integration.

As used herein, the term “expression vector” may include a plasmid vector, a cozmid vector, an episomal vector, a viral vector, etc., preferably the viral vector. The viral vector used herein may include a vector derived from retrovirus, lentivirus, adenovirus, adeno-associated virus, herpes simplex virus, Sendai virus, etc., preferably a lentiviral vector, but is not limited thereto.

As used herein, the term “reporter gene” refers to a gene attached to a gene of interest in order to determine the expression pattern of the gene of interest. Depending on uses, there are various types of reporter gene, including, but not limited to, β-galactosidase gene, β-glucuronidase (GUS) gene, green fluorescent protein (GFP) gene, luciferase gene, chloramphenicol acetyltransferase (CAT) gene, cyan fluorescent protein (CFP) gene, yellow fluorescent protein (YFP) gene, and the like, preferably green fluorescent protein (GFP) gene and luciferase gene.

As used herein, the term “cartilage disease” refers to a disease caused by injury to cartilage, cartilage tissues and/or joint tissues (synovial membrane, articular cavity, subchondral bone, etc.) due to mechanical stimulation or inflammatory reaction, and includes cartilage injury diseases. Such cartilage disease may include degenerative arthritis, rheumatoid arthritis, fracture, damage to muscle tissues, plantar fasciitis, humerus epicondylitis, calcification myositis, nonunion of fracture, or a joint damage caused by injury, but is not limited thereto.

In the present invention, the Col2a1 promoter may be represented by a sequence of SEQ ID NO: 1, but is not limited thereto.

In the present invention, the Col2a1 promoter enhancer may be also represented by a sequence of SEQ ID NO: 2, but is not limited thereto.

In the present invention, the recombinant expression vector may be also represented by a sequence of SEQ ID NO: 3, but is not limited thereto.

In the present invention, the recombinant expression vector may be also represented by a sequence of SEQ ID NO: 4, but is not limited thereto.

In addition, the variants of the sequence may be also included within the scope of the present invention, and may specifically include a sequence having at least 70% homology, more preferably at least 80% homology, much more preferably at least 90% homology, and most preferably at least 95% homology with the above sequence. The “% of sequence homology” for polynucleotide may be identified by comparing two optimally aligned sequences with a comparison region, and a part of the polynucleotide sequence in the comparison region may include an addition or deletion (i.e., a gap) compared to a reference sequence for the optimal alignment of two sequences (not including an addition or deletion).

In the present invention, the Col2a1 promoter may be human type II collagen promoter.

In addition, in the present invention, the Col2a1 promoter and the Col2a1 promoter enhancer may be derived from human mesenchymal stem cells, but are not limited thereto.

The mesenchymal stem cells may include the adult stem cells derived from mammals and the adult stem cells may be derived from the adult stem cells of all the tissues. For example, the adult stem cells may be selected from bone marrow-derived, cord blood-derived, blood-derived, liver-derived, skin-derived, gastrointestinal-derived, placenta-derived, nerve-derived, adrenal-derived, epithelial-derived, uterine-derived ones, and the like.

In addition, the recombinant expression vector according to the present invention may be prepared by using a lentiviral vector.

In another aspect of the present invention, the present invention may provide a transformed cell line for screening cartilage disease treatment agents transformed using the recombinant expression vector.

In the present invention, the transformed cell line may be a human cartilage cell line.

In addition, the transformation may be performed in the present invention by using a lentivirus prepared by using the recombinant expression vector according to the present invention.

In addition, the transformed cell line in the present invention may be C28/I2-EC2P-fLuc-CN5 of accession number KCLRF-BP-00456.

The present inventors have named the novel cell line as C28/I2-EC2P-fLuc-CN5 and deposited the same with accession number KCLRF-BP-00456 to the Korean Cell Line Research Foundation.

In still another aspect of the present invention, the present invention may provide a method for screening cartilage disease treatment agents, the method comprising: treating the transformed cell line with a candidate drug; and measuring a level of expression or activity of a reporter gene in the transformed cell line treated with the candidate drug.

In the present invention, the reporter gene may be a gene encoding luciferase or green fluorescent protein (GFP).

In the present invention, the method may further include determining that a drug having a higher level of expression or activity of the reporter gene measured above is regarded to have a more excellent regenerative activity for damaged cartilage.

In still another aspect of the present invention, the present invention may provide a composition for treating cartilage diseases, containing 2-anthraquinonecarboxylic acid or derivatives thereof as an effective ingredient.

In the present invention, “2-anthraquinonecarboxylic acid” is called 9,10-dioxoanthracene-2-carboxylic acid, anthraquinone-2-carboxylic acid, 2-carboxyanthraquinone, etc., under the IUPAC names, and is known as a material having anti-inflammatory efficacy.

As used herein, the term “derivative” refers to a compound in which a functional group is introduced, substituted, oxidized, reduced, etc., in the 2-anthraquinonecarboxylic acid, so as to change the compound to the extent that the structure and properties of the parent are not drastically changed. Thus, there is no limit to the kind of the derivative.

In the present invention, the composition may treat cartilage diseases through cartilage regeneration.

In addition, in the present invention, the cartilage disease may be selected from the group consisting of degenerative arthritis, rheumatoid arthritis, fracture, damage to muscle tissues, plantar fasciitis, humerus epicondylitis, calcification myositis, nonunion of fracture, and joint damage caused by injury.

Furthermore, the present invention may provide a method for treating cartilage diseases, the method including administering a composition containing 2-anthraquinonecarboxylic acid or derivatives thereof as an effective ingredient to an individual.

In another aspect of the present invention, the present invention may provide a use of a composition containing 2-anthraquinonecarboxylic acid or derivatives thereof as an effective ingredient for treating cartilage diseases.

In still another aspect of the present invention, the present invention may provide a use of 2-anthraquinonecarboxylic acid or derivatives thereof for producing a drug used for treating cartilage diseases.

Hereinafter, the present invention will be described in detail through preferred Examples for better understanding of the present invention. However, the following Examples are provided only for the purpose of illustrating the present invention, and thus the present invention is not limited thereto.

### Example 1

### Example 1. Preparation of Lentiviral Vector Containing Type II Collagen Fusion Promoter and Reporter Gene

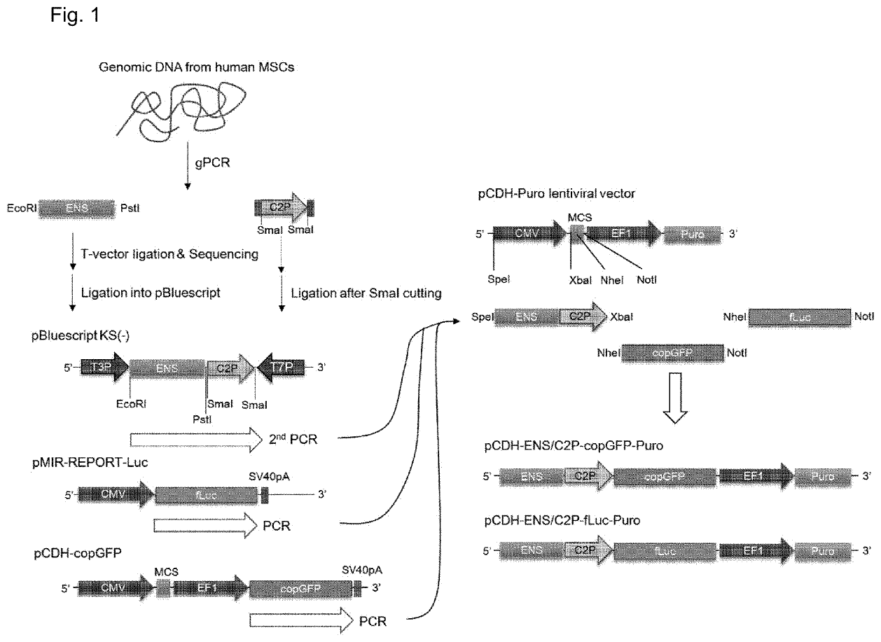

As shown in FIG. 1, Col2a1 promoter enhancer (ENS) DNA and core promoter C2P DNA, which correspond to +2127 to +2842 bp of the first intron of the type 2 collagen gene amplified from genomic DNA of human mesenchymal stem cells through a polymerase chain reaction (PCR), are first linked to T-vector to confirm the authenticity of each base sequence, and then linked to pBluescript KS(−) shuttle vector, so as to complete a fused human type II synthetic collagen promoter.

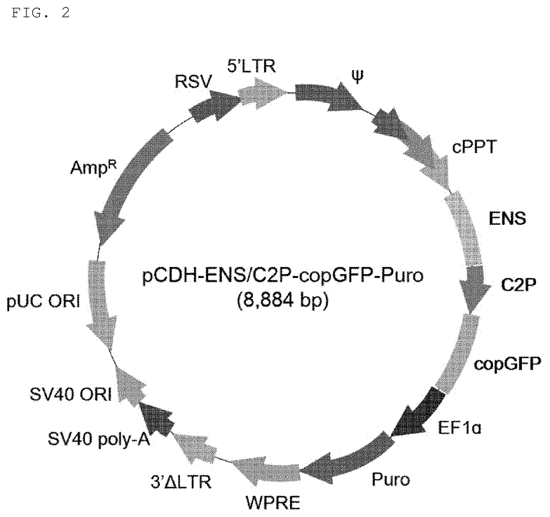

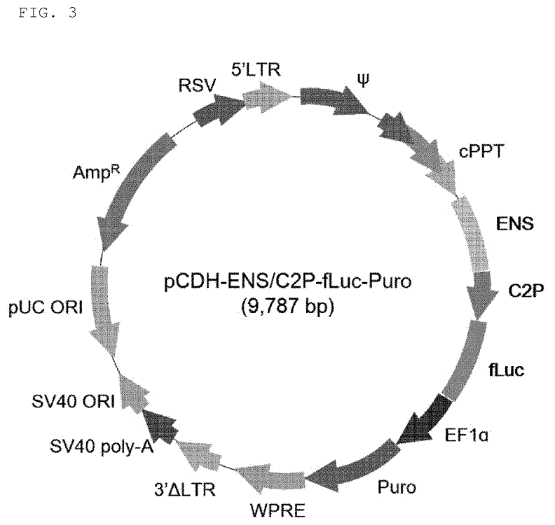

And, the fusion promoter was re-amplified from the vector through a single PCR reaction, and the resulting product was inserted into a SpeI-XbaI position present in pCDH-Puro vector and thus exchanged with CMV promoter. Then, the final target pCDH-ENS/C2P-copGFP-Puro (FIG. 2) and pCDH-ENS/C2P-fLuc-Puro (FIG. 3) lentiviral vectors were completed by linking the luciferase gene amplified from pMIR-REPORT-Luc vector or the copGFP gene amplified from pCDH-copGFP vector at a NheI-NotI position, which is the next restriction enzyme section of the vector.

### Example 2. Confirmation of Induced Cartilage Tissue-Specific Expression of Type II Collagen Fusion Promoter

The copGFP-Puro lentivirus was synthesized by using the pCDH-ENS/C2P-copGFP-Puro vector and 293FT cell prepared in above Example 1, and a mesenchymal stem cell, which is a pre-differentiated cell, and a chondrocyte cell line C28/I2, which is a differentiated cell, were infected with the virus, and thus the expression of GFP was confirmed under a fluorescence microscope.

In this case, a vehicle without a vector was used as a transformed negative control group, and a pECFP non-viral vector capable of inducing the expression of GFP in both cells before and after differentiation was used as a positive control group.

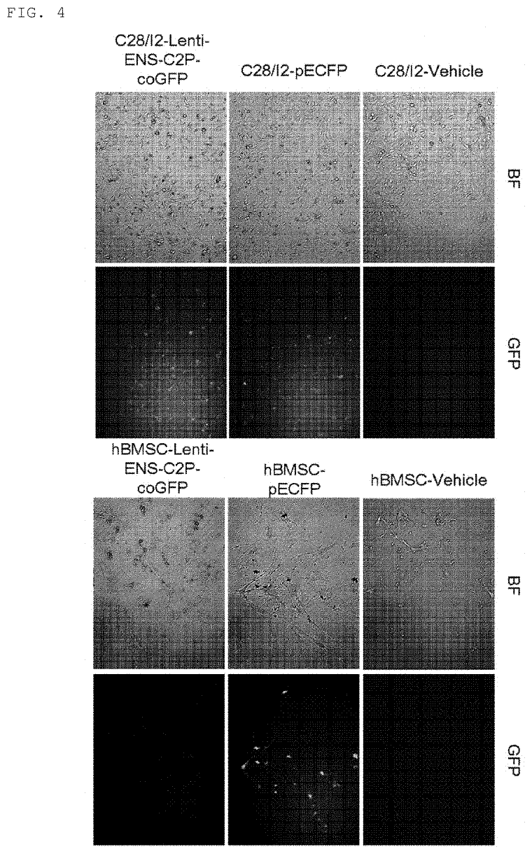

Consequently, as shown in FIG. 4, it was confirmed that GFP fluorescence is observed in both the mesenchymal stem cells and the C28/I2 cell line, which are transformed by using the pECFP vector, while GFP fluorescence is detected only in the C28/I2 cartilage cell line, if the two cells are transformed by using the copGFP-Puro lentivirus. The above results mean that the human-derived type II collagen fusion promoter prepared in Example 1 is induced to specifically express in chondrocytes only and thus works as intended.

### Example 3. Screening of Mono Clone of Human-Transformed Cartilage Cell Line Capable of Expressing Luciferase Specifically to Chondrocyte

The fLuc-Puro lentivirus was synthesized by using the vector and 293FT cell prepared in above Example 1, and the virus was used for infection of human cell line C28/I2. After infecting the cells at a virus concentration of MOI=30 for 12 hours, the cells were further treated with DMEM/F12 culture fluid containing 4 μg/ml of puromycin antibiotics at an interval of two to three days until the cells no longer die and grow normally, thereby establishing a polyclonal transformed C28/I2 cell population.

After that, the finally established cell population was cultured with a concentration of antibiotics reduced to 1 μg/ml, and the cell population was used and further subjected to a monoclonal screening process for about one month through a 96-well plate cell dilution method, thereby obtaining 14 types of monoclonal cell lines. As each monoclonal cell line has a different degree of reaction to the drug, each cell was treated with Kartogenin or TD-198946, which is known to have an excellent effect of cartilage regeneration and differentiation, so as to secure an optimal monoclone, and then the reaction of each clone was observed. First, the cells were seeded at 1×10⁴ cells into each well of a 96-well plate a day before being treated with drugs, and all the cells were subjected to starvation in serum-free media for seven hours on the following day, and then were cultured with the addition of complete DMEM/F12 media so that the concentration of each drug reached 10 μM.

After culturing for 24 hours, all the culture media were removed, and new complete DMEM/F12 media containing 150 μg/ml of D-luciferin were added to each well and reacted at room temperature for five minutes. Then, the luciferase activity was analyzed by using the luminescence analysis option of TECAN SPARK multileader.

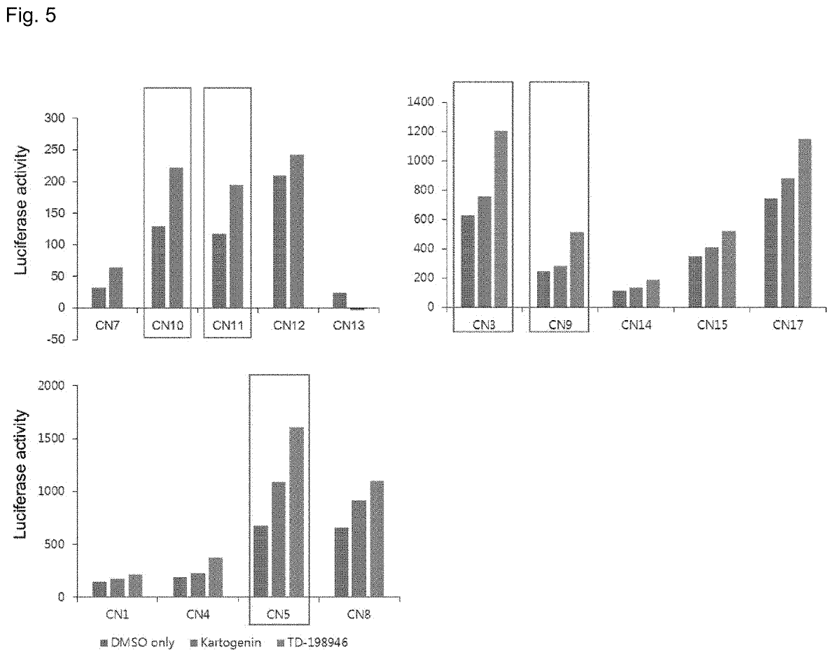

Consequently, as shown in FIG. 5, it was confirmed that all the clones show a reaction to the two drugs compared to a vehicle negative control group (DMSO only), and it was also confirmed that out of the clones Clone Number (CN) 5 cell line shows a distinct difference in a degree of reactivity to each drug while showing a high luciferase activity compared to other clones, and thus was established as C28/I2-EC2P-fLuc-CN5 and deposited (with the accession number KCLRF-BP-00456) to the Korean Cell Line Research Foundation.

### Example 4. Verification of Efficacy of Cartilage Treatment Drugs Using C28/I2-EC2P-fLuc-CN5 Cell Line

As mentioned above, the C28/I2-EC2P-fLuc-CN5 cell line established in above Example 3 was seeded into a 96-well plate, and its cellular reaction was observed when being treated with Kartogenin and TD-198946 known as cartilage treatment drugs at each concentration, in which the concentration of each drug was 0 to 50 μM.

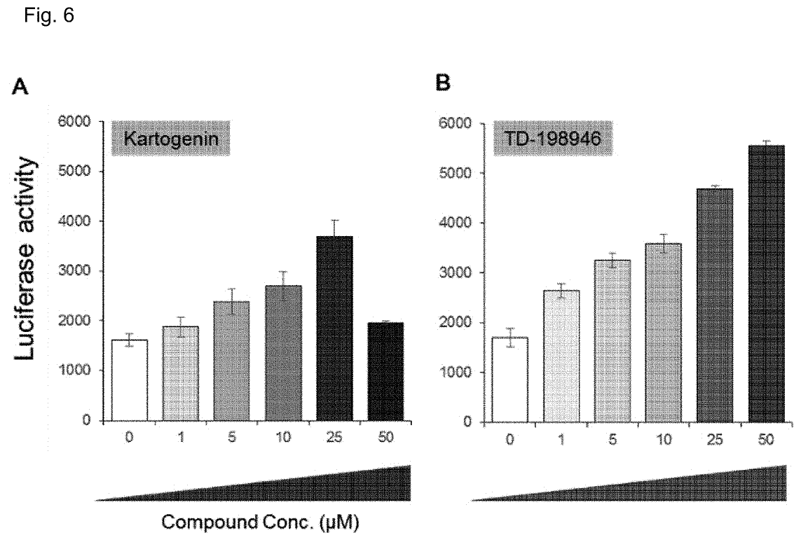

Consequently, as shown in FIG. 6, it was found that the luciferase activity is increased as the concentration of both drugs is increased. In particular, it was confirmed that the activity of TD-198946 is higher than that of Kartogenin, suggesting that the efficacy of the two drugs, which have not been compared yet with each other, may be directly compared by using the present screening cellular system for effective drugs for cartilage treatment.

In addition, unlike TD-198946, which continuously increases the luciferase activity, it was confirmed that Kartogenin decreases the luciferase activity at a level of 50 μM, indirectly suggesting that Kartogenin has cytotoxicity at a high concentration.

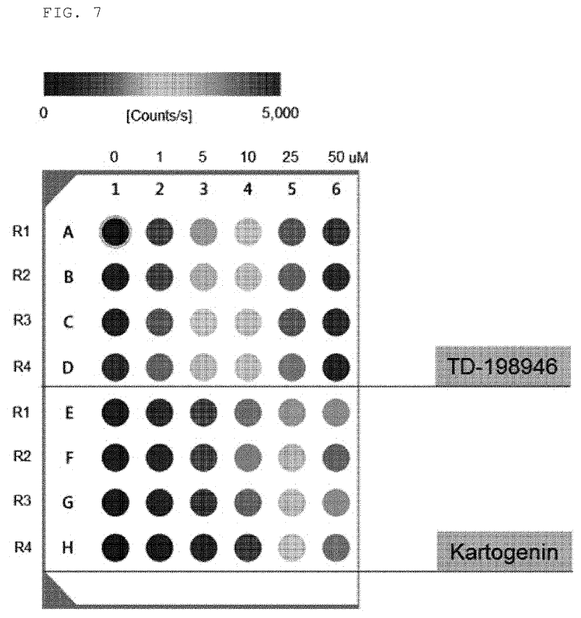

Furthermore, depending on the options of the measuring device, the above results may be imaged so that an analyst can easily evaluate the drug activity. FIG. 7 shows an image of results of treatment repeatedly four times with each drug, in which a more red color shows a more excellent activity of the drug on cartilage treatment, and the error range of the repeated results between respective wells is not large, suggesting that a total of 96 drugs may be screened with the current system without a repeated test.

### Example 5. Screening of Novel Cartilage Treatment Agent and Verification of Effect by Using Screening Cellular System for Effective Drugs for Cartilage Treatment

The screening system for cartilage disease treatment agents according to the present invention was used to screen a material having an excellent cartilage regeneration efficacy out of various anti-inflammatory natural materials, thereby verifying the screening system according to the present invention and finding 2-anthraquinonecarboxylic acid (hereinafter referred to as “2-AQCA”), which is a novel material having an excellent cartilage regeneration efficacy. The specific experimental method is as follows.

### 5.1. Search for Cartilage Regeneration Efficacy of Anti-Inflammatory Natural Material

The 37 kinds of natural materials with known anti-inflammatory effects to be used in this example were selected based on the anti-inflammatory natural compound library (ChemFace, China). The types of the compound were shown in table 1 below.

TABLE 1

| No. | Compound Name |
|---|---|
| 1 | Tannic acid |
| 2 | Glycyrrhizic acid |
| 3 | Ginsenoside compound K |
| 4 | Arctiin |
| 5 | Madecassic acid |
| 6 | Oleanolic acid |
| 7 | Wilforlide A |
| 8 | Peimine |
| 9 | Hydrocortisone |
| 10 | Neochlorogenic acid |
| 11 | Bavachinin |
| 12 | Berberine |
| 13 | Sinomenine |
| 14 | Lithospermoside |
| 15 | Isobavachalcone |
| 16 | Quercetin |
| 17 | Ellagic acid |
| 18 | Embelin |
| 19 | Nonivamide |
| 20 | Shikonin |
| 21 | Wogonin |
| 22 | Apigenin |
| 23 | Alnustone |
| 24 | Indirubin |
| 25 | 4-(p-Biphenylyl)-3-hydroxybutyric acid |
| 26 | 2-anthraquinonecarboxylic acid |
| 27 | Atractylenolide II |
| 28 | Resveratrol |
| 29 | Methyl syringate |
| 30 | Alpha-caryophyllene |
| 31 | Zingerone |
| 32 | Methyl gallate |
| 33 | Chelidonic acid |
| 34 | Paeonol |
| 35 | Methyl cinnamate |
| 36 | Pyrogallol |
| 37 | 4-hydroxybenzyl alcohol |

All the experimental groups for the verification of the screening system according to the present invention and the search for novel cartilage regeneration materials were prepared by dissolving the same in dimethyl sulfoxide (DMSO) at the same concentration of 20 mM. The cells to be analyzed were seeded and fixed into a white-bottom 96-well plate, which was a plate exclusively used for luminescence analysis, at an initial concentration of 10,000 cells per well 24 hours before the experiment. On the following day, the cells were subjected to starvation in serum-free culture fluid (DMEM/F12 media) for seven hours. After that, while the culture fluid was replaced with the serum culture fluid [DMEM/F12, 10% FBS, 1% penicillin, and streptomycin], the experimental groups on natural products at each 20 mM were diluted 1/400, treated repeatedly three times, and further cultured for 48 hours. In this case, the final concentration of the treated natural products was 50 μM, and DMSO without the above materials was used as a negative control group, whereas Kartogenin and TD-198946, which were used in Examples 3 and 4 for the verification test of the screening system according to the present invention as natural materials with known cartilage regeneration efficacy, were used at the same concentration as a positive control.

### 5.2. Selection of Active Material for Cartilage Regeneration

After further culturing the cells to be analyzed, which were treated with natural materials in above 5.1, for 48 hours, all the culture fluid was removed and replaced with a new culture fluid containing D-luciferin at a final concentration of 150 μg/ml and reacted at room temperature for five minutes. Then, the degree of light emission from each cell was measured by using the luminescence analysis function of the TECAN SPARK multi-reader. After searching each material with the screening system for cartilage disease treatment agents according to the present invention, it was found that TD-198946 shows a cartilage regeneration activity higher than that of Kartogenin, and thus a final material estimated to have a cartilage regeneration activity was selected in accordance with the criterion of having the cartilage regeneration activity equal to or higher than that of Kartogenin.

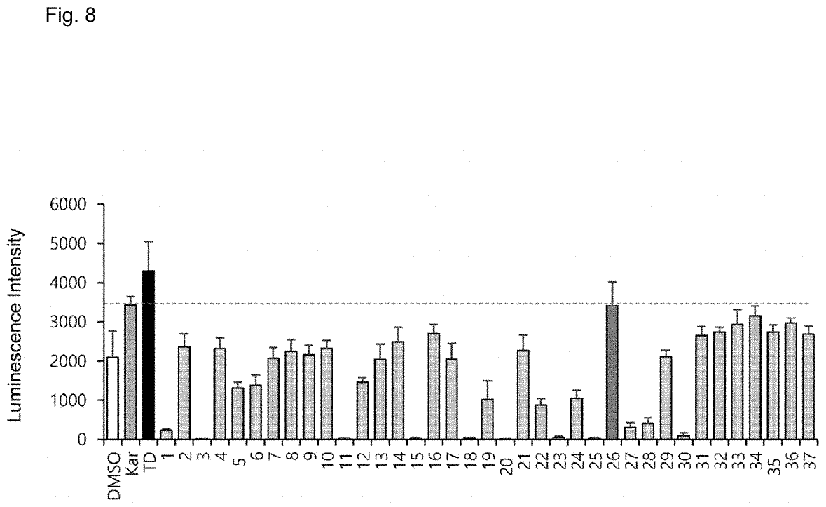

Consequently, as shown in FIG. 8, it was confirmed that 2-AQCA, which is a material No. 26 represented by Formula 1 below, complies with the above conditions. Accordingly, the cartilage regeneration activity, the cytotoxicity, and the expression patterns of cartilage differentiation markers were verified for 2-AQCA as follows.

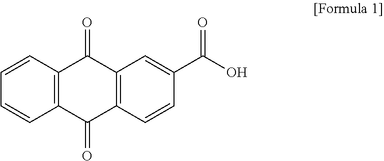 [SMILES: O=C(O)c1ccc2c(c1)C(=O)c1ccccc1C2=O]

### 5.3. Verification of Cartilage Regeneration Activity of 2-AQCA at Each Concentration

By using the screening system for cartilage disease treatment agents according to the present invention, the pattern of changes in cartilage regeneration activity according to the treatment concentration of 2-AQCA was compared with the activity of Kartogenin, a positive control group of natural material. The process of preparing and analyzing the cells was the same as above, except that the cells were treated at the final concentration of the test materials at 0, 3.125, 6.25, 12.5, and 25 μM, respectively, and the final concentration of DMSO was 0.25% so that the experimental conditions are the same in all the experimental groups.

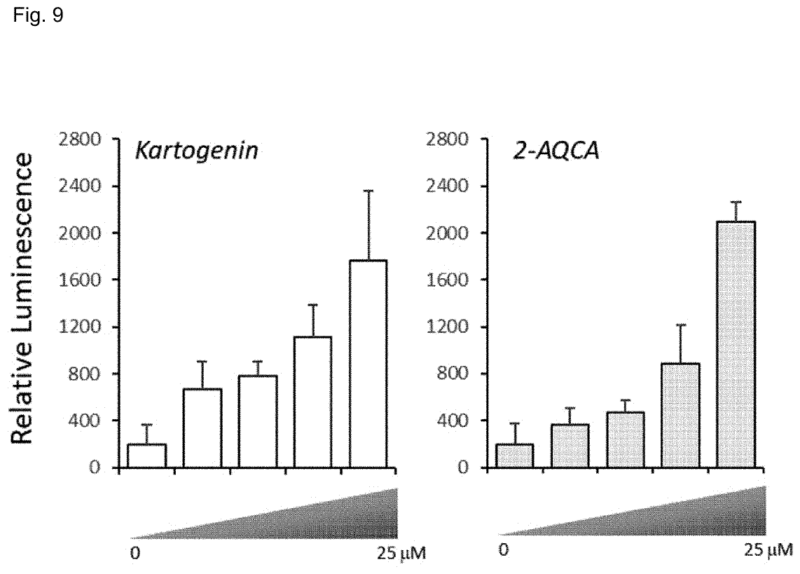

Consequently, as shown in FIG. 9, when treated with 2-AQCA at each concentration, it was confirmed that an increase in the activity is similar to that of Kartogenin used as a positive control group. Even when being treated at a concentration of less than 25 μM, it was confirmed that the activity is somewhat lower than that of Kartogenin, but the cartilage regeneration activity is slightly higher than that of Kartogenin at 25 μM.

### 5.4. Verification of Cytotoxicity of 2-AQCA

To confirm the effect of 2-AQCA on inhibiting a cell growth rate and the presence of toxicity, the cells to be analyzed (C28/I2 cell line and chondrocytes isolated from osteoarthritis patients) were seeded and fixed at an initial concentration of 10,000 cells per well in a 96-well plate 24 hours before the experiment, and the cells were subjected to starvation in serum-free culture fluid for seven hours on the following next day. After that, while the culture fluid was replaced with a serum culture fluid containing 10% FBS, the experimental groups were treated repeatedly three times with Kartogenin and 2-AQCA at each concentration and further cultured for 48 hours. The cells were treated with the test materials at each concentration of 0, 3.125, 6.25, 12.5, and 25 μM, which had been used to verify the effect of treatment at each of the concentrations. Similarly, in order to equalize the experimental conditions, the treatment was performed so that the final concentration of DMSO was 0.25% in all the experimental groups. In 48 hours later, all the culture fluid was removed, the cells were washed repeatedly twice with 200 μl of Dulbecco's Phosphate-Buffered Saline (DPBS) per well, and 200 μl of the culture fluid containing 1/10 volume of EZ-Cytox (DoGen Inc., Korea) was added, and then further cultured under the condition of 37° C. and 5% CO2 for one hour. Then, the absorbance of each cell was measured at 450 nm by using a TECAN SPARK multi-reader.

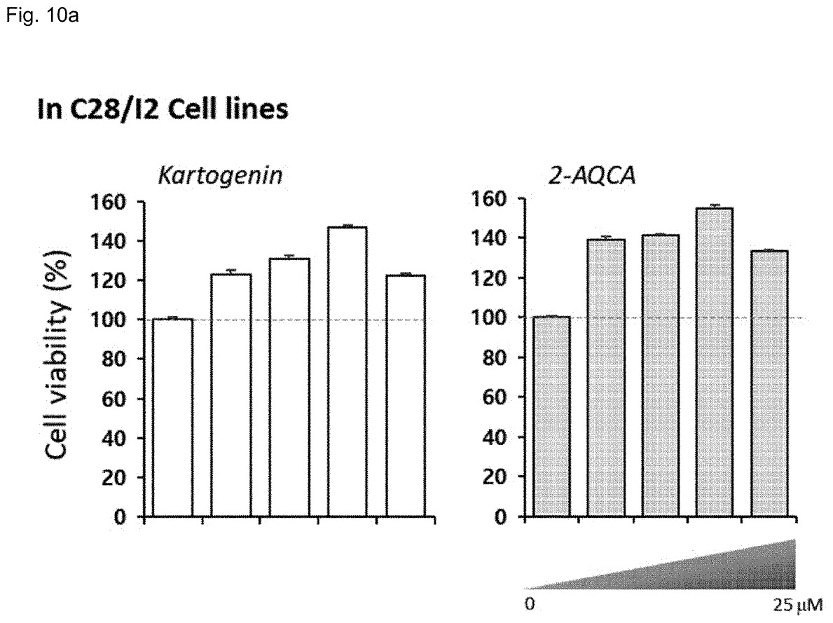

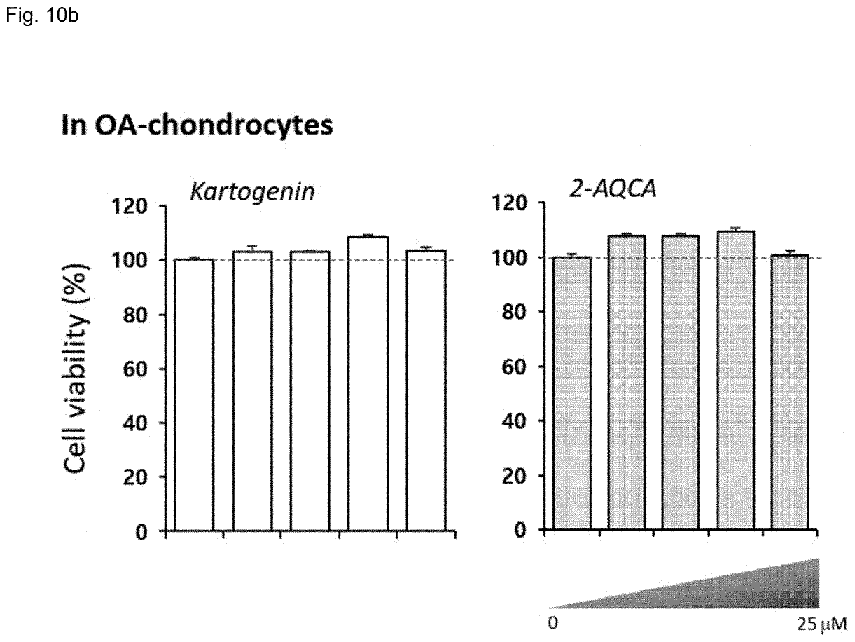

Consequently, as shown in FIGS. 10a and 10b, when the human chondrocyte cell line C28/I2 was treated with Kartogenin and 2-AQCA at each concentration, the effect of inhibiting a cell growth rate due to toxicity was not found. Rather, it was confirmed for both natural materials that the cell growth rate is increased by up to 47% or more compared to that of a negative control group (DMSO treatment group) (FIG. 10A). Comparing the cell growth rates of both treatment materials, it was found that 2-AQCA shows a synergistic effect in the cell growth rate about 11% higher in the concentration range of 6.25 μM and about 8.2% higher in the concentration range of 12.5 μM than that of Kartogenin. Even when the chondrocytes isolated from osteoarthritis patients were treated with Kartogenin and 2-AQCA at each concentration by the same method, it was found that there is no effect of inhibiting a cell growth rate due to toxicity of the test materials, and it was also found in the concentration range of 12.5 μM or less that the result of increasing a cell growth rate is similar to that of the experiment on chondrocyte cell lines with 8 to 9% or less.

### 5.5. Comparison of Expression Patterns of Cartilage Differentiation Markers by 2-AQCA Treatment

C28/I2 cell lines were subjected to micromass culture in order to compare and examine the change in the expression of Col2a1 and Sox9, which are the representative cartilage differentiation markers, and Col10, which is a cartilage hypertrophy marker, by treatment with the complex functional natural material 2-AQCA during long-term culture for inducing cartilage differentiation. For this purpose, the C28/I2 cells cultured for three days were collected and a cell fluid was prepared at a concentration of 2×10⁷ cells/ml, and then 10 μl of cells were taken and injected into the center of a 24-well plate, and fixed in an incubator of 37° C., 5% CO2 for two to three hours until all the cells were attached to the bottom. When all the cells were fixed, 500 μl of serum culture fluid (DMEM/F12, 10% FBS, 1% penicillin, and streptomycin) was added and further cultured for seven hours or more. After a cell mass was stably formed on the following day, the culture fluid was replaced with a new culture fluid, to which each natural material was added, so as to perform a culture for inducing differentiation for a total of 14 days while replacing the culture fluid at an interval of two to three days. In this case, a treatment concentration of natural materials was 10 μM, the level at which the cell growth rate was highest, and 0.1% DMSO was used to minimize cytotoxicity.

After completing the micromass culture for 14 days, RNA was isolated from each cell pellet by using TRIzol™, and cDNA was synthesized from the isolated RNA. The synthesized cDNA was compared and analyzed for expression of the corresponding gene through a real-time quantitative polymerase chain reaction (Rq-PCR) by using a specific primer for each gene. The primer sequences of the corresponding genes are as follows:

| | Col2a1, Forward, |
|---|---|
| (SEQ ID NO: 5) | |
| | 5′-AACCAGATTGAGAGCATCCG-3′; |
| | Col2a1, Reverse, |
| (SEQ ID NO: 6) | |
| | 5′-ACCTTCATGGCGTCCAAG-3′; |
| | Sox9, Forward, |
| (SEQ ID NO: 7) | |
| | 5′-ACTTGCACAACGCCGAG-3′; |
| | Sox9, Reverse, |
| (SEQ ID NO: 8) | |
| | 5′-CTGGTACTTGTAATCCGGGTG-3′; |
| | Col10a1, Forward, |
| (SEQ ID NO: 9) | |
| | 5′-ACGATACCAAATGCCCACAG-3′; |
| | Col10a1, Reverse, |
| (SEQ ID NO: 10) | |
| | 5′-GTACCTTGCTCTCCTCTTACTG-3′; |
| | Gapdh, Reverse, |
| (SEQ ID NO: 11) | |
| | 5′-ACATCGCTCAGACACCATG-3′; |
| | Gapdh, Reverse, |
| (SEQ ID NO: 12) | |
| | 5′-TGTAGTTGAGGTCAATGAAGGG-3′; |

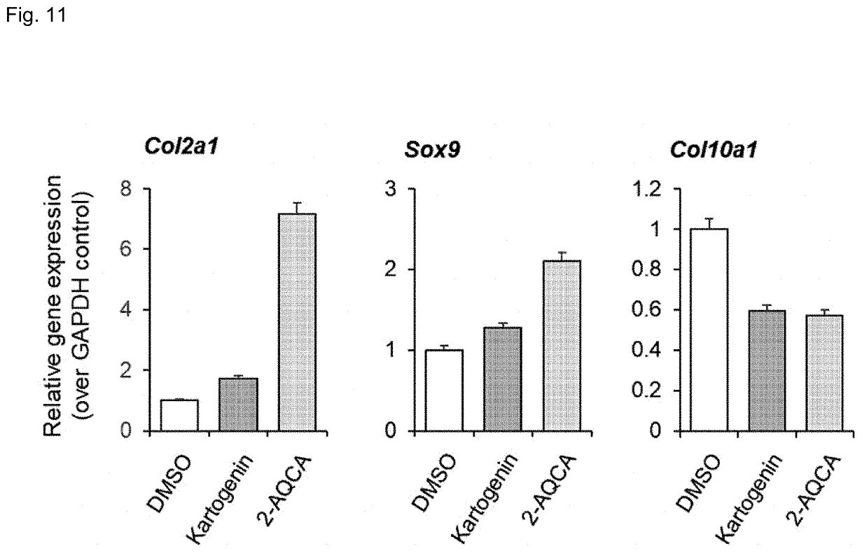

As a result of performing Rq-PCR analysis, as shown in FIG. 11, it was confirmed that when being treated with 2-AQCA, Col2a1, which is the most important marker of cartilage regeneration, shows an expression rate about 4.2 times higher than that of the group treated with Kartogenin, which is the positive control group, and shows an expression rate 7.6 times higher than that of the negative control group not treated with natural products. In addition, it was confirmed that when being treated with Kartogenin, Sox9, which is a major transcription factor for inducing the synthesis of Col2a1 in chondrocytes, shows an insignificant increase compared to the negative control group, whereas the group treated with 2-AQCA shows a genetic expression at least 2.1 times higher. Finally, it was confirmed that the Col10a1 gene, which induces thickening of chondrocytes to induce apoptosis, shows a decrease in expression about 40% compared to the negative control group in both groups treated with Kartogenin or 2-AQCA. Summarizing the above results, it could be confirmed that 2-AQCA is a novel material capable of promoting cartilage differentiation and regeneration while inhibiting thickening of chondrocytes, and shows an efficacy much more excellent than Kartogenin, which is known as a cartilage regeneration material.

In summary, when using the cell system for screening effective drugs for cartilage treatment according to the present invention through a series of experiments disclosed through the above examples, it is possible to efficiently select a cartilage treatment agent having a cartilage regeneration effect, and thus it was proved that a novel cartilage regeneration material, 2-AQCA, exhibits a more excellent cartilage regeneration effect compared to previously known cartilage regeneration materials.

The above description of the present invention is for illustrative purposes only, and those skilled in the art to which the present invention pertains can easily understand that the present invention may be easily transformed into other specific forms without changing the technical spirit or essential features of the present invention. Thus, it is to be understood that the exemplary embodiments described above are illustrative in all aspects and are not contrived to limit the scope of the present invention.

### INDUSTRIAL APPLICABILITY

According to the present invention, the screening system for cartilage disease treatment agents is expected to be very useful in the pharmaceutical industry as it can compare the effects of various drugs on cartilage disease treatment with each other, and also evaluate the optimal treatment concentration and the presence or absence of indirect cytotoxicity. In addition, 2-anthraquinonecarboxylic acid, which has been found through the screening system of the present invention, shows an excellent efficacy of regenerating cartilage, and thus may be widely used as various therapeutic agents for cartilage diseases.

1. A recombinant expression vector for screening cartilage disease treatment agents, comprising:
Col2a1 promoter (C2P),
Col2a1 promoter enhancer (ENS), and
a reporter gene.

2. The recombinant expression vector of claim 1, wherein the Col2a1 promoter is represented by a sequence of SEQ ID NO: 1.

3. The recombinant expression vector of claim 1, wherein the Col2a1 promoter enhancer is represented by a sequence of SEQ ID NO: 2.

4. The recombinant expression vector of claim 1, wherein the Col2a1 promoter is human type II collagen promoter.

5. The recombinant expression vector of claim 1, wherein the Col2a1 promoter and the Col2a1 promoter enhancer are derived from human mesenchymal stem cells.

6. The recombinant expression vector of claim 1, wherein the recombinant expression vector is prepared by using a lentiviral vector.

7. The recombinant expression vector of claim 1, wherein the recombinant expression vector is represented by a sequence of SEQ ID NO: 3.

8. The recombinant expression vector of claim 1, wherein the recombinant expression vector is represented by a sequence of SEQ ID NO: 4.

9. A transformed cell line for screening cartilage disease treatment agents, which is transformed with a lentivirus prepared by using the recombinant expression vector of claim 1.

10. The transformed cell line of claim 9, wherein the transformed cell line is a human-derived cartilage cell line.

11. The transformed cell line of claim 9, wherein the transformed cell line is C28/12-EC2P-fLuc-CN5 of accession number KCLRF-BP-00456.

12. A method for screening cartilage disease treatment agents, the method comprising:
treating the transformed cell line of claim 9 with a candidate drug; and
measuring a level of expression or activity of a reporter gene in the transformed cell line treated with the candidate drug.

13. The method of claim 12, further comprising determining a drug having a higher level of measured expression or activity of the reporter gene as a drug having a more excellent regenerative activity for damaged cartilage.

14. A composition for treating cartilage diseases, comprising 2-anthraquinonecarboxylic acid or derivatives thereof as an effective ingredient.

15. The composition of claim 14, wherein the composition treats cartilage diseases through cartilage regeneration.

16. The composition of claim 14, wherein the cartilage disease is selected from the group consisting of degenerative arthritis, rheumatoid arthritis, fracture, damage to muscle tissues, plantar fasciitis, humerus epicondylitis, calcification myositis, nonunion of fracture, and joint damage caused by injury.

17. A method for treating cartilage diseases, the method comprising:
administering a composition containing 2-anthraquinonecarboxylic acid or derivatives thereof as an effective ingredient to an individual.

18. A use of the composition of claim 14, comprising 2-anthraquinonecarboxylic acid or derivatives thereof as an effective ingredient, for treating cartilage diseases.

19. A method for screening cartilage disease treatment agents, the method comprising:
treating the transformed cell line of claim 10 with a candidate drug; and
measuring a level of expression or activity of a reporter gene in the transformed cell line treated with the candidate drug.

20. A method for screening cartilage disease treatment agents, the method comprising:
treating the transformed cell line of claim 11 with a candidate drug; and
measuring a level of expression or activity of a reporter gene in the transformed cell line treated with the candidate drug.
